(19) United States
(12) Patent Application Publication
(10) Pub. No.: US 2023/0039272 A1
(43) Pub. Date: Feb. 9, 2023

(54) COMPOSITION FOR IMPROVED ENERGY, FOCUS AND CLARITY

(71) Applicant: Gwella Mushrooms Inc., Toronto (CA)

(72) Inventor: Daniel C. SANDERS, Little Rock, AR (US)

(21) Appl. No.: 17/733,623

(22) Filed: Apr. 29, 2022

Related U.S. Application Data
(60) Provisional application No. 63/220,639, filed on Jul. 12, 2021.

(51) Int. Cl. A61K 36/48; A61K 31/4015; A61K 31/7076; A61K 31/522; A61K 31/675; A61P 25/00; A61K 36/258; A61K 36/16; A61K 36/9068; A61K 36/07; A61K 36/062; A61K 31/197

(52) U.S. Cl. CPC A61K 36/48; A61K 31/4015; A61K 31/7076; A61K 31/522; A61K 31/675; A61P 25/00; A61K 36/258; A61K 36/16; A61K 36/9068; A61K 36/07; A61K 36/062; A61K 31/197

(57) ABSTRACT

A composition comprising: a racetam; an extract of Mucuna Pruriens; S-Adenosyl methionine; and caffeine; useful for improvement of alertness, physical endurance, sleep patterns, memory, perceptual acuity, and/or for increasing energy.

### CROSS REFERENCE TO RELATED APPLICATIONS

This application depends from and claims priority to U.S. Patent Application No. 63/220,639 filed Jul. 12, 2021, the entire contents of which are incorporated herein by reference.

### FIELD OF THE INVENTION

The invention relates to a pharmaceutical or nutraceutical composition for improved energy, focus and clarity.

### BACKGROUND OF THE INVENTION

### Microdosing of Psychedelic Compounds

The microdosing of psychedelic compounds has gained tremendous popularity over the last decade, with many notable public figures extolling its virtues. Microdosing psychedelic compounds involves consuming very low, sub-psychedelic doses of psychedelic compounds, such as psilocybin—containing mushrooms (“Magic Mushrooms”) or lysergic acid diethylamide (“LSD”). Both anecdotal reports and more rigid (though often preliminary) scientific studies have confirmed certain desirable effects of microdosing psychedelic compounds, including improved mood, visual and aural acuity, perception, focus, and decreased anxiety (Anderson et al., “Psychedelic microdosing benefits and challenges: an empirical codebook” Harm Reduction Journal 16:43(2019)). Other reported effects include a heightened sense of smell, colour and depth of visual acuity, improved mood, an ability to reach “flow state” with ease, increased productivity and enjoyment in tasks, enhanced creativity, an increased alertness and sense of being, and improved perception of and empathy with others.

Several companies are now in the process of conducting pre-clinical and clinical studies on microdosing psilocybin, for example, for a variety of beneficial effects. However, since psilocybin (and most, if not all, psychedelic substances) are controlled substances or illegal drugs in many countries, these studies are constrained by the current drug approval and prescription pathway, and thus typically limited to defined medical indications, such as anxiety, depression, and obesity. This is unfortunate, since much of the anecdotal evidence of benefits come from healthy individuals, often at the top of their game, who report that microdosing improve wellness, acuity, alertness, etc., in an already healthy individual (i.e. helps them go from 0 to 6, rather than from −6 to 0).

Because psychedelics such as psilocybin are controlled or illegal in many countries, and because there is a perception of harm (largely because of their illegality, but also because of their historic, recreational uses), there is a hesitancy in the general population to their use. Also, the pathway to approval will likely be more complex and/or political than the typical approval of a new chemical entity.

Accordingly, there is a desire and need in the market for compounds engineered to mimic a neurological experience similar to that of microdosing of a psychedelic mushroom, using legal, obtainable, “functional food” ingredients.

### Nootropics

Nootropics, sometimes referred to as “smart drugs” and “cognitive enhancers”, are drugs, supplements and other substances that are believed to improve cognitive function, particularly executive functions, memory, creativity, or motivation in healthy individuals. Many racetams (a class of drugs that share a pyrrolidone backbone), are considered to be nootropics. Racetams have very few adverse effects and low toxicity.

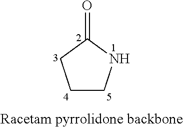

Certain racetams are believed to be nootropics; these include aniracetam, piracetam, oxiracetam, pramiracetam, and phenylpiracetam.

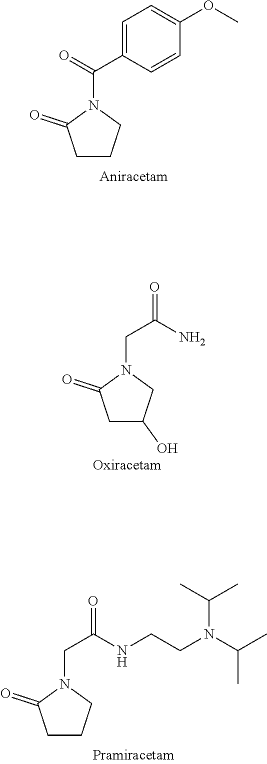 [SMILES: COc1ccc(C(=O)N2CCCC2=O)cc1 ; NC(=O)CN1CC(O)CC1=O ; CC(C)N(CCNC(=O)CN1CCCC1=O)C(C)C]

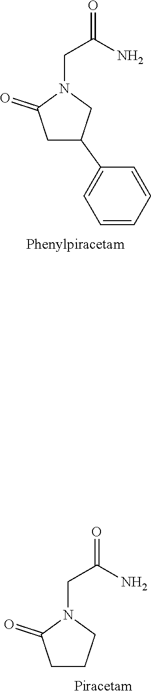 [SMILES: NC(=O)CN1CC(c2ccccc2)CC1=O ; NC(=O)CN1CCCC1=O]

Oxiracetam, for example, has been shown to be save, even at high doses for long periods of time. It is believed to have mild stimulant effect. Patients with mild to moderate dementia, administered oxiracetam, experienced higher test scores on logical performance, attention, concentration, memory and special orientation, and, importantly, improved quality of life scale (Bottini et al., Acta Neurol Scand 1992 September; 86(3):237-241). Oxiracetam was found to improve learning of animals in pre-clinical studies (Krylova et a., Farmakol Toksikol January-February 1991; 54(1):14-16; PMID 1860490) and to improve step-down retention performance and acquisition performance in active-avoidance situations (indicating improved learning and memory) (Mondadori et al, Clin Neuropharmacol. 1986; 9 Suppl 3:S27). Oxiracetam is widely commercially available (for example, from Health Naturals (www.healthbynaturals.com).

Piracetam is currently marketed as a dietary supplement for treatment for myoclonus and as a cognitive enhancer. There are some studies suggesting piracetam may be useful for treatment of dementia or cognitive problems, though these largely remain inconclusive. It has also been suggested for lowering depression, reducing anxiety, improving memory, and treatment of vertigo. It is believed to promote microcirculation (including to the brain), by diminishing erythrocyte adhesion to vascular wall endothelium.

### N-Phenylacetyl-L-Prolylglycine Ethyl Ester

N-phenylacetyl-L-prolylglycine ethyl ester, also called omberacetam and sold under the brand name Noopept™ is also believed to be a nootropic, and preliminary studies have shown it may have cognition restoring effect in animal models of Alzheimer's Disease, and a neuroprotective effect in an animal model of Alzheimer's Disease, including protection of the impairments of cell viability, calcium homeostasis, Reactive Oxygen Species levels, mitochondrial function, tau phosphorylation, and neurite outgrowth caused by Amyloid Beta exposure (a known animal model of Alzheimer's Disease) (Ostrovskaya et al, J Biomed Sci 2014 Aug. 6; 21(1):74). It is believed to boost levels of brain-derived neurotrophic factor and is commonly available as a synthetic “smart drug” supplement (see for example, from Health Naturals (www.healthbynaturals.com).

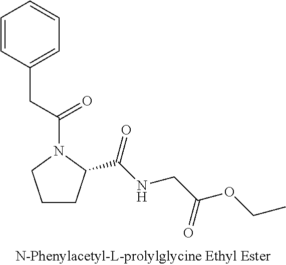 [SMILES: CCOC(=O)CNC(=O)[C@@H]1CCCN1C(=O)Cc1ccccc1]

Panax Ginseng

Ginseng, or the root of Panax ginseng Meyer, Family Araliaceae, is one of the most well-known oriental medicinal herbs. It has been widely used as a herbal remedy for a wide variety of diseases and disorders. Ginseng is available in a natural-dried form (known as white ginseng), as well as red ginseng, which is steamed prior to drying. Panax ginseng is distinguished from other plants sometimes referred to as ginseng, such as American ginseng, which has a different composition and different pharmacological activity.

Panax ginseng is known to contain a wide variety of pharmacological components, including a series of tetracyclic triterpenoid saponins (known as ginsenosides), polyacetylenes, polyphenolic compounds, and acidic polysaccharides. Certain ginsenosides are only found in red ginseng as they are derived from the hydrolysis of saponins during the steaming stage.

Ginseng has been described in literally thousands of articles, both in traditional Chinese medicines and in western peer reviewed publications. The majority of these relate to ginseng's immunity activity—it has been reported to maintain homeostasis of the immune system, to enhance resistance to illness or microbial attacks through the regulation of the immune system. It has also been reported to improve male reproductive function and aid with erectile dysfunction, reduce inflammation, have beneficial antioxidant properties, help with eczema, improve memory, behavior and mood, improve “calmness”, mental performance, and math skills, decrease mental fatigue. Studies have suggested positive effects on brain function and behavior in patients with Alzheimer's Disease. Panax ginseng has been shown to help fight fatigue and promote energy, and may help in regulation of blood sugar. It has even been suggested that taking ginseng regularly may stave off certain types of cancer.

Panax ginseng is widely commercially available, for example, from Now Foods (available at iherb.com).

### Ginger Root

Ginger (Zingiber officinale) is a flowering plant whose rhizome, ginger root, is widely used both as a spice and as a medicine and supplement. Ginger may help alleviate nausea and vomiting, and is used to prevent sea sickness. It is believed to have anti-inflammatory and antioxidant effects, and has been suggested for treatment of osteoarthritis. It appears to reduce blood sugar in patients with type 2 diabetes, and may help reduce cholesterol levels. Ginger may help enhance brain function, improve reaction time, and working memory. It has been suggested that ginger may act as an antibacterial, especially against oral bacteria and help with gingivitis and periodontitis.

Ginger root can be purchased at most grocery stores, and is available in an extract (for example, from Now Foods, available at www.iherb.com).

### Ginkgo Biloba

Ginkgo biloba is a tree species whose fan shaped leaves are farmed for extract. Ginkgo biloba is rich in flavonoids and terpenoids. Ginkgo biloba extract is believed to improve circulation, and is best known as a potential treatment for dementia, claudication, Alzheimer's Disease, and fatigue. It is believed to improve blood flow and as such may help with blood pressure problems, erectile dysfunction, premenstrual syndrome, asthma, vascular dementia (atherosclerotic vascular disease—related dementia), leg pain caused by clogged arteries, and macular degeneration. It has been suggested to improve blood flow to the brain, aid cognitive function, boost memory, sharpen thinking, aid with chronic schizophrenia, ADHD, autism, and generalized anxiety disorder. Most of the suggested effects of Ginkgo biloba have been found to be anecdotal, inconclusive, or controversial. Ginkgo biloba supplements are widely available in the form of extracts, and pills (for example, from Now Foods, available at www.iherb.com).

### Mucuna Pruriens Extract

Mucuna pruriens is a tropical legume also known as velvet bean, monkey tamarind, cowhage, and Lyon bean. Though primarily used as a food or fodder plant, its seeds have been used as a medicine in Ayurvedic medicine and in Unani medicine. It has been used as an antitoxin for snakebites from cobra, viper, and other snakes, and has been suggested as an alternative treatment for Alzheimer's Disease due to its high content of L-dopa. Its seeds are believed to alleviate neurotoxicity induced by Parkinson's disease. Mucuna Pruriens has been suggested to be useful for anxiety as well as joint and muscle pain. It is available for example from Paradise Herbs, available at www.iherb.com.

### Vicia Faba

Vicia faba is also known as fava bean. It is primarily used as a food and is a large part of the eastern Mediterranean diet. Vicia faba comprises c-aminobutyric acid (GABA), which can act as a neurotransmitter, transfer nerve impulse, and may aid in regulating blood pressure, heart rate, palliates pain, and apprehensiveness. It may remedy a number of diseases, for example, by inhibiting topoisomerase through its polyphenolic compounds. V. faba seeds comprise phenolic antioxidant activity, and contain levo-dihydroxy phenylalanine (L-DOPA), which may be useful in treating Parkinson's disease. V. Faba also has anti-microbial activity, and contains trypsin inhibitor, chymotrypsin inhibitor, chitinase, wyerone, and wyerone epoxide and as such may also have antifungal activity. It may also have anticancer and antidiabetic activity. Cooked V. faba is used as a medication for a cough and the flowers are used for dressing inflammations, warts, and burns. (Sathya Prabhu and Devi Rajeswari, International Food Research Journal 25(4): 1332-1340 (August 2018)).

### SAM-e

S-Adenosyl methionine (SAM-e) is a cosubstrate involved in methyl group transfers, transsulfuration, and aminopropylation.

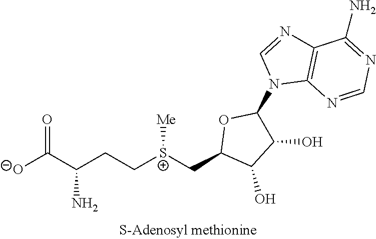 [SMILES: C[S@@+](CC[C@H](N)C(=O)[O-])C[C@H]1O[C@@H](n2cnc3c(N)ncnc32)[C@H](O)[C@@H]1O]

SAM-e has been studied for use in the mitigation of osteoarthritis pain, as an anti-cancer treatment, for depression, anxiety, back pain, fibromyalgia, and for alcoholic cirrhosis of the liver and other liver diseases. It is available as a dietary supplement in many countries. It is commercially available, for example, from Now Foods at iherb.com.

### Cordycep Militaris

Cordycep militaris is a species of parasitic fungi that grows on the larvae of insects. It can also be grown in rice. It is available as a supplement in powder and extract form, and is thought to improve energy levels and endurance, treat chronic fatigue and fatigue in general, boost sexual function, relieve auto-immune disease, and reduce inflammation. Cordyceps is thought to increase the body's function of ATP, and therefore may boost exercise performance and endurance, including tolerance to high intensity exercise, and even improve a person's VO2 max. It may have anti-tumor effects, and appears to mimick the action of insulin to decrease blood sugar level. Cordycep militaris has been used for the treatment of arrhythmia, decrease risk of heart failure, and have heart-protective effects. It is believed to have immunomodulation effects (Lee, et al., Pharmacol 30 Nov. 2020). It is commercially available, for example, from manufacturer Now Foods at www.iherb.com.

### Cordycep Sinensis

Cordycep sinesis, also known as Ophiocordyceps sinesis, is a species of fungi closely related to the mushroom. It is a parasitic fungus that grows on caterpillar. It is available as a supplement in powder and extract form, and is thought to improve energy levels and endurance, treat chronic fatigue, boost sexual function, relieve auto-immune disease, improve breathing, circulation, and reduce inflammation. It has a long history of use as a traditional Chinese medicine, as well as traditional Tibetan medicine. It is used in traditional healers of Sikkim for the treatment of a large amount of illnesses, claiming it improves energy, appetite, stamina, libido, endurance, and sleeping patterns (Panda and Swain, J Ayurveda Integr Med 2011, January-March; 2(1):9-13). It contains cordyceptin, a derivative of adenosine which is believed to have selective cytotoxicity against leukemic cells. It is commercially available, for example, from manufacturer Now Foods at www.iherb.com.

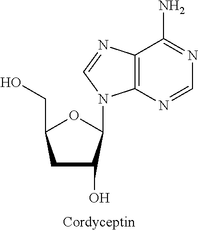

### Lion's Mane

Lion's Mane is a mushroom commonly used and available in powder, extract, and pill form, as a nutritional supplement, believed to have neuroprotection, neurotrophic activity, to improve cognitive function, to assist with neurodegenerative diseases, relieve anxiety and depression, and reduce inflammation. Lion's Mushrooms can also be enjoyed as a food—raw, cooked, or steeped as a tea. Lion's mane contains hericenones and erinacines, thought to stimulate the growth of brain cells, protect against degenerative brain disease and progressive memory loss, and reduce symptoms of memory loss and prevent neuronal damage caused by amyloid-beta plaques, which accumulate int eh brain during Alzheimer's disease. It appears to boost mental functioning, treats mild cognitive impairment, and may promote nerve growth. There have been preliminary studies suggesting it can relieve mild symptoms of depression and anxiety, helps regenerate brain cells, and has anti-inflammatory effects. It may help speed recovery from injuries to the brain or spinal cord, and decrease inflammation and reduce the size of stroke-related brain injury. Lion's mane is believed to lower blood sugar levels in both normal and diabetic individuals, in part by blocking the activity of alpha-glucosidase. It is also believed to reduce inflammation and oxidative stress. It is commercially available, for example, from producer Mushroom Wisdom on www.iherb.com

### L-theanine

L-theanine, also called L-γ-glutamylethylamide or N5-ethyl-L-glutamine, is an amino acid analog of L-glutamate and L-glutamine.

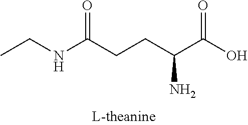

It is found in certain types of mushroom, as well as in green and black tea. It is available as a nutritional supplement in pill or tablet form, and is considered Generally Recognized as Safe by the Food and Drug Administration. It is thought to ease anxiety, stress, and reduce insomnia, particularly in people experiencing stressful situations and in people living with schizophrenia or schizoaffective disorder. It is believed to increase focus and attention, promote faster simple reaction time, faster numeric working memory reaction time, and improved sentence verification accuracy, particularly when paired with caffeine. It may help with increased blood pressure after specific mental situations, and, while it is non-drowsy, was found to improve sleep quality, especially among people with ADHD or schizophrenia. L-theanine has also been reported to raise levels of brain serotonin, dopamine, and GABA, with possible improvement in specific memory and learning tasks. It is commercially available, for example, from supplement manufacturer Now Foods, on www.iherb.com.

### Caffeine

Caffeine is a central nervous system stimulant, and the world's most widely consumed psychoactive drug. It prevents adenosine-induced drowsiness by blocking the action of adenosine on the adenosine receptor, and stimulates certain portions of the autonomic nervous system.

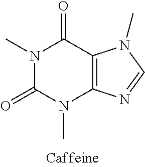

It is most commonly found in coffee bean and coffee-based drinks, though it is found in tea, cola, and energy beverages worldwide. It may confer a modest protective effect against Parkinson's disease. Its most common use is as a stimulant that reduces fatigue and drowsiness. It generally improves reaction time, wakefulness, concentration, and motor co-ordination. Concurrent caffeine and L-theanine use appears to promote alertness, attention, and task switching. It has also been shown to improve athletic performance, improving sprint performance, delaying muscle fatigue, and increasing basal metabolic rates. Caffeine is readily available also in sustained release, extended release, and slow release formulations, for example, extended release caffeine 60, which slowly releases the caffeine over 60 minutes.

### SUMMARY OF THE INVENTION

According to one aspect of the invention is provided a composition comprising: a racetam; an extract of Mucuna Pruriens; S-Adenosyl methionine; and caffeine.

In certain embodiments, the composition further comprises second racetam.

In certain embodiments, the composition further comprises N-phenylacetyl-L-prolylglycine ethyl ester.

In certain embodiments, the composition further comprises any one or more of: Panax ginseng; Ginkgo biloba; ginger root; Cordyceps militaris; Lion's Mane; L-Theanine; and Cordyceps Sinesis.

In certain embodiments, the racetam is aniracetam, piracetam, oxiracetam, pramiracetam, or phenylpiracetam.

In certain embodiments, the racetam is oxiracetam.

In certain embodiments, the second racetam is piracetam.

In certain embodiments, the composition further comprises psilocin or psilocybin.

In certain embodiments, the composition comprises oxiracetam, N-phenylacetyl-L-prolylglycine ethyl ester, Panax ginseng, ginger root, Mucuna Pruriens Extract, S-adenosyl methionine, Cordyceps Militaris, Lion's Mane, L-theanine, caffeine, and Cordyceps Sinesis.

In certain embodiments, the composition comprises, as a weight percentage of active ingredients: 20-30% oxiracetam, 0.4-0.8% Noopept, 2-4% Panax ginseng, 6-8% ginger root, 10-15% Mucuna Pruriens extract, 2-5% Sam-E, 8-12% Cordyceps Militaris, 12-18% Lion's Mane, 3-5% L-theanine, 6-10% delayed release caffeine, and 8-12% Cordyceps Sinensis.

In certain embodiments, the composition comprises, as a weight percentage of active ingredients: about 25% oxiracetam, about 0.6% Noopept, about 3% Panax ginseng, about 7% ginger root, about 13% Mucuna Pruriens extract, about 3.5% Sam-E, about 10% Cordyceps Militaris, about 15% Lion's Mane, about 4% L-theanine, about 8% delayed release caffeine, and about 10% Cordyceps Sinensis.

In certain embodiments, the composition comprises, per dose unit: 37.5-375 mg of oxiracetam; 0.9-9 mg of N-phenylacetyl-L-prolylglycine ethyl ester; 4.3-44 mg of panax ginseng; 10.5-105 mg of ginger root; 18-190 mg of Mucuna Pruriens extract; 5-53 mg of Sam-E; 14-145 mg of Cordyceps Militaris; 22-225 mg of Lion's Mane; 6-60 mg of L-theanine; 12-120 mg of caffeine; 14-25-150 mg of Cordyceps Sinesis.

In certain embodiments, the composition comprises, per dose unit: 50-187.5 mg of oxiracetam; 1.2-4.5 mg of N-Phenylacetyl-L-prolylglycine ethyl ester; 5.8-22 mg of panax ginseng; 14-52.5 mg of ginger root; 25-94 mg of Mucuna Pruriens extract; 7-25 mg of Sam-E; 19-72 mg of Cordyceps Militaris; 30-112 mg of Lion's Mane; 8-30 mg of L-theanine; 16-60 mg of caffeine; and 20-72 mg of Cordyceps Sinesis.

In certain embodiments, the composition comprises, per dose unit: about 62.5 mg of oxiracetam; about 1.5 mg of N-phenylacetyl-L-prolylglycine ethyl ester; about 7.3 mg of panax ginseng; about 17.5 mg of ginger root; about 31.25 mg of Mucuna Pruriens extract; about 8.75 mg of Sam-E; about 23.75 mg of Cordyceps Militaris; about 37.5 mg of Lion's Mane; about 10 mg of L-theanine; about 20 mg of caffeine; and about 23.75 mg of Cordyceps Sinesis.

In certain embodiments, the caffeine is a delayed release caffeine.

In certain embodiments, the delayed release caffeine is a delayed release caffeine 60 or a delayed release caffeine 80.

In certain embodiments, the composition further comprises non-medicinally active ingredients selected from fillers, excipients, stabilizers, sweeteners, compacting agents, solubilizing agents, anti-flocculants, and gelling agents.

In certain embodiments, the composition is in the form of a gummy, a capsule, or a tablet.

According to a further aspect of the invention is provided a method of improving alertness, physical endurance, sleep patterns, memory, perceptual acuity, and/or increasing energy in an individual, comprising administering an effective amount of the composition.

According to a further aspect of the invention is provided the use of the composition for improvement of alertness, physical endurance, sleep patterns, memory, perceptual acuity, and/or for increasing energy.

### BRIEF DESCRIPTION OF FIGURES

FIG. 1 shows the effects of administration of an embodiment of the present composition on healthy individuals.

### DETAILED DESCRIPTION

Unless otherwise explained, all technical and scientific terms used herein have the same meaning as commonly understood by one of ordinary skill in the art to which this disclosure belongs. Patent applications, patents, and publications referred to herein to assist in the understanding of the aspects described are herewith incorporated by reference in their entirety.

In understanding the scope of the present application, the articles “a”, “an”, “the”, and “said” are intended to mean that there are one or more of the elements. Additionally, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives.

It will be understood that any aspects describing as “comprising” certain components may also “consist of” or “consist essentially of”, wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention.

All ranges given herein include the end of the ranges and also any intermediate range points, whether explicitly stated or not.

Terms of degree such as “substantially”, “about” and “approximately” as used herein mean a reasonable amount of deviation of the modified term such that the end result is not significantly changed. These terms of degree should be construed as including a deviation of at least +/−5% of the modified term if the deviation would not negate the meaning of the word it modifies.

Although methods and materials similar or equivalent to those described herein can be used in the practice or testing of this disclosure, suitable methods and materials are described below. The abbreviation “e.g.” is used herein to indicate a non-limiting example. The word “or” is intended to include “and” unless the context clearly indicates otherwise.

Described is a composition comprising a unique formulation of ingredients, in specific ratio ranges. The composition is useful as a dietary supplement and/or a nutraceutical.

When taken, the composition provides a variety of advantageous effects, which may simulate the neurological experience of the microdosing of a psychedelic drug, such as psilocybin. The composition is safe for human consumption, preferably avoids the use of controlled or illegal substances (such as psilocybin) and may provide many beneficial effects. These effects may include: an energy boost, which may be a sustained energy boost lasting up to 6-8 hours; an enhancement of physical focus, clarity and precision; improved physical performance including for aerobic and non-aerobic exercise and competition, such as squash, racquetball, tennis, hockey, basketball, and other sports involving either or preferably both physical performance and mental acuity; improve overall performance baseline; improved mental acuity; improved visual acuity; improved auditory acuity; improved empathy and “connection” with other individuals; decreased anxiety; improved mental focus, including concentration, conscious awareness, mindfulness, and increased engagement and attentiveness; improved hand-eye co-ordination; better sleep; better focus; heightened flow state activation; improved mood state, including happiness, well-being, peace, calm, optimism, and reductions in depressive symptoms; improved health behaviours; improved creativity, including divergent thinking, curiosity, shifting perspectives, and openness; improved self-efficacy, including motivation, ambition, productivity, confidence, sense of agency; improved self-care, including introspection, meditation, and other behaviours facilitating mental health; improved energy, including alertness, wakefulness, and stimulation; social benefits, including extraversion, empathy, sense of connection, and verbal fluency; cognitive benefits, including cognitive enhancement, clarity of thought, and memory; reduced anxiety including social-anxiety; enhanced senses; improved cardiovascular endurance; reduced migraines and/or headaches; stress reduction; reduced sensitivity to trauma; reduced substance dependence; improved mood; and better short term memory. The effects may be felt without the typical side effects of stimulants, such as the “jitters” or a “crash”.

The composition may be taken orally, as a pill, powder, extract, or in a “gummy” form. It may be incorporated into a food or drink, or a lozenge or lollipop. In the form of a lozenge or lollipop, it may be administered orally, interbucally, or sublingually. In the form of a powder, it may be taken orally, or by means of internasal administration. The composition may be vaporized and inhaled.

The composition comprises: a racetam, for example, aniracetam, piracetam, oxiracetam, pramiracetam, and/or phenylpiracetam; N-phenylacetyl-L-prolylglycine ethyl ester; extract of Mucuna Pruriens; S-Adenosyl methionine; and caffeine. Optionally, the composition may further comprise any one or more of: Panax ginseng; Ginkgo biloba; ginger root; Cordyceps militaris; Lion's Mane; L-Theanine; and Cordyceps Sinesis. In some embodiments, the N-phenylacetyl-L-prolylglycine ethyl ester may be substituted with a second racetam. In some embodiments, the caffeine is a delayed release caffeine, such as delayed release caffeine 60 or delayed release caffeine 80.

In certain embodiments, the composition comprises, per dosage: 37.5-375 mg, for example, 50-187.5 mg, for example about 62.5 mg of oxiracetam; 0.9-9 mg, for example 1.2-4.5 mg, for example about 1.5 mg of N-Phenylacetyl-L-prolylglycine ethyl ester; 4.3-44 mg, for example, 5.84-22 mg, for example about 7.3 mg of panax ginseng; 10.5-105 mg, for example, 14-52.5 mg, for example, about 17.5 mg of ginger root; 18-190 mg, for example 25-94 mg, for example about 31.25 mg of Mucuna Pruriens extract; 5-53 mg, for example 7-25 mg, for example about 8.75 mg of Sam-E; 14-145 mg, for example 19-72 mg, for example about 23.75 mg of Cordyceps Militaris; 22-225 mg, for example, 30-112 mg, for example about 37.5 mg of Lion's Mane; 6-60 mg, for example 8-30 mg, for example about 10 mg of L-theanine; 12-120 mg, for example, 16-60 mg, for example 20 mg of Delayed Release Caffeine 60; and 14.25-150 mg, for example 20-72 mg, for example, 23.75 mg of Cordyceps Sinesis.

In certain embodiments, the composition comprises, as weight percent of active ingredients: 20-30%, preferably about 25% oxiracetam, 0.4-0.8, preferably about 0.6% Noopept, 2-4%, preferably about 3% Panax ginseng, 6-8%, preferably about 7% ginger root, 10-15, preferably about 13% Mucuna Pruriens Extract, 2-5%, preferably about 3.5% Sam-E, 8-12%, preferably about 10% Cordyceps Militaris, 12-18%, preferably about 15% Lion's Mane, 3-5%, preferably about 4% L-theanine, 6-10%, preferably about 8% delayed release caffeine, and 8-12, preferably about 10% Cordyceps Sinensis.

The combination of ingredients in the composition may provide unexpected effects that are enhanced over what would be expected from the ingredients acting alone or in combination. The effects may be additive, or synergistic. The specific ratio of ingredients is also tailored such that it may provide unexpected additional benefits. For example, the ingredients may act in concert with one another, or complement one another, in a herebefore unexpected manner.

The composition may also comprise non-medicinal ingredients, such as fillers, excipients, stabilizers, sweeteners, compacting agents, solubilizing agents, anti-flocculants, gelling agents, and the like.

### EXAMPLES

### Example 1: Manufacture of Gummy Dosages

Gummy dosages are increasingly popular. Typically, these are gelatin-based gels or gums, which are in the form of a teddy bear or other shape. These are often referred to as “gummy bears” or the like, and are of similar consistency and density as the popular children's candies.

12 Compositions of the invention are manufactured utilizing the ingredients and quantities as described in Table 1, and the methodology described in below. Briefly, water is heated to 70 degrees C. and a suitable amount unflavored gelatin is added and mixed until dissolved. Corn syrup, pre-heated to 110 degrees C., is added to the gelatin mixture. The active ingredients as described in Table 1, in fine powder form, are then added and solubilized. Any desired wet ingredients, for example, flavors, preservatives, and dyes, or fillers, excipients, stabilizers are then added and the resulting solution is mixed until completely dissolved. The solution is decanted into 100 silicone gelatin molds and the gelatin allowed to cure at room temperature or light refrigeration for 12 to 18 hours or until set. The resultant gummies are removed from the molds and optionally tossed in sugar and/or another desired outer coating, such as citric acid or tartaric acid (for “sours”).

TABLE 1

| | 1 | 2 | 3 | 4 | 5 | 6 | 7 | 8 | 9 | 10 | 11 | 12 |
|---|---|---|---|---|---|---|---|---|---|---|---|---|
| Ingredient | (g) | | | | | | | | | | | |
| Oxiracetam | 6.2 | 6.2 | | 6.2 | 5.0 | 3.7 | 37.5 | 18.8 | | 10.3 | 5.7 | 6.5 |
| Pramiracetam | 0 | 0 | 6.2 | 0 | 0 | 0 | 0 | 0 | 15 | 0 | 0 | 0 |
| Piracetam | 0 | 6.2 | | 0 | 0 | 0 | 0 | 0 | 10 | 0 | 0 | 0 |
| Noopept | 0.15 | 0 | 0.15 | 0.15 | 0.12 | 0.09 | 0.9 | 0.5 | 0 | 0.21 | 0.11 | 0.3 |
| Panax | 0 | 0 | 0.73 | 0.73 | 0.584 | 0.44 | 4.4 | 2.2 | 0 | 0.54 | 2.00 | 1.5 |
| Ginseng | | | | | | | | | | | | |
| Ginkgo | 0 | 0 | 0 | 0 | 1.5 | 0 | 0 | 0 | .8 | 0.53 | 0 | 0.2 |
| Biloba | | | | | | | | | | | | |
| Ginger Root | 0 | 0 | 0 | 1.75 | 0 | 1.0 | 10.5 | 5.3 | 2 | 2.0 | 4.3 | 1.3 |
| Mucuna | 3.125 | 3.125 | 3.125 | 3.125 | 2.5 | 1.9 | 19.0 | 9.4 | 3 | 4.0 | 7.1 | 3.7 |
| Pruriens | | | | | | | | | | | | |
| Extract | | | | | | | | | | | | |
| Sam-E | 0.875 | 0.875 | 0.875 | 0.875 | 0.7 | 0.55 | 5.3 | 2.6 | 1 | 2.0 | 1.4 | 0.7 |
| Cordyceps | 0 | 0 | 0 | 2.375 | 1.9 | 1.40 | 14.3 | 7.1 | 2.5 | 4.0 | 1.0 | 5.4 |
| Militaris | | | | | | | | | | | | |
| Lion's Mane | 0 | 0 | 0 | 3.75 | 3.0 | 2.20 | 22.5 | 11.2 | 4 | 5.0 | 10.3 | 3.7 |
| L-theanine | 0 | 0 | 0 | 1.0 | 0.8 | 0.6 | 6 | 3 | 1 | 0 | 1.5 | 3.0 |
| Delayed | 0 | 2.0 | 2.0 | 2.0 | 1.6 | 0.6 | 12 | 6 | 1 | 1 | 4.0 | 7.0 |
| Release | | | | | | | | | | | | |
| Caffeine 60 | | | | | | | | | | | | |
| Delayed | 2.0 | 0 | 0 | 0 | 0 | 0.6 | 0 | 0 | 1 | 1 | 0 | 0 |
| Release | | | | | | | | | | | | |
| Caffeine 80 | | | | | | | | | | | | |
| Cordyceps | 0 | 0 | 2.375 | 2.375 | 1.9 | 1.40 | 14.3 | 7.2 | 2 | 3.0 | 5.2 | 2.1 |
| Sinesis | | | | | | | | | | | | |

### Example 2: Manufacture of Capsules

12 compositions of the invention are manufactured utilizing the active ingredients and quantities as described in Table 1. Briefly, the active ingredients as described in Table 1 are obtained as or rendered into fine powders, and mixed in a blender to homogeneity. Methylcellulose fiber is added as required, to decrease “deadspace” and to harbor a consistent capsule. A powder mixer is used to homogenize the powder, which is then transferred to a capsule filling unit. 100 hard capsules are filled, then tumble coated, using conventional methods.

### Example 3: Administration of Compound

The compounds made in Examples 1 and 2 are administered, daily to individuals, for one month. Individuals self-report various beneficial effects, including a decrease in challenging experiences associated with ADHD, improved energy, alertness, calmness, improved physical endurance, improved sleep, cognitive enhancement, improved mood, creativity, balanced energy without jitters or a crash, increased focus and achieving a flow state-like experience significantly more than placebo controls.

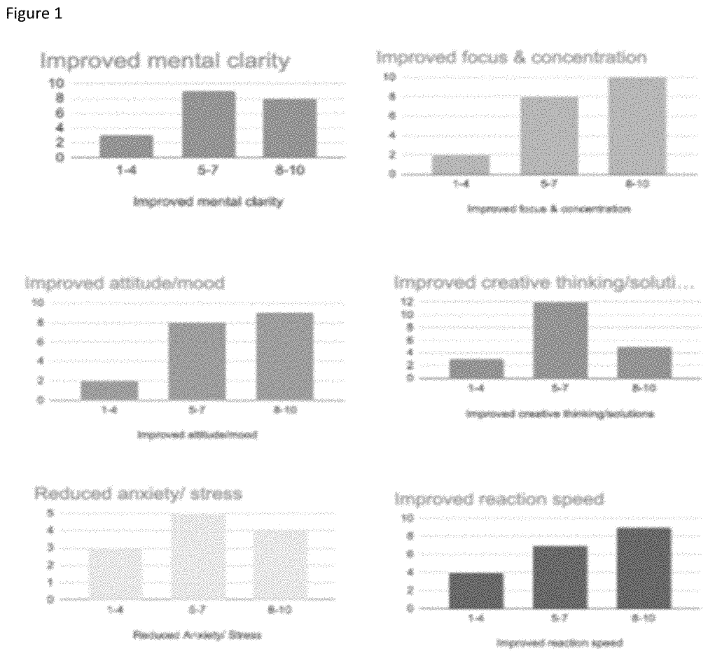

In product testing and customer feedback involving 22 participants (including both men and women and individuals between the ages of 18-55 in both the United States and Canada), individuals were asked to rate on a scale of 1-10, how much the administration of compound 4 of Table 1 improved their focus, mood, reaction speed, mental clarity, creativity, and reduced anxiety. The scores were tabulated and shown in FIG. 1. Most participants reported improved mental clarity, improved focus and concentration, improved attitude/mood, improved creative thinking/solution, reduced anxiety and stress, and improved reaction speed.

The above disclosure generally describes the present invention. Although specific terms have been used herein, such terms are intended in a descriptive sense and not for purposes of limitation.

All publications, patents and patent applications cited above are herein incorporated by reference in their entirety to the same extent as if each individual publication, patent or patent application was specifically and individually indicated to be incorporated by reference in its entirety.

Although preferable embodiments of the invention have been described herein in detail, it will be understood to those skilled in the art that variations may be made thereto without departing from the spirit of the invention or the scope of the appended claims.

1. A composition comprising: a racetam; an extract of Mucuna Pruriens; S-Adenosyl methionine; and caffeine.

2. The composition claim 1 further comprising a second racetam.

3. The composition of claim 1 further comprising N-phenylacetyl-L-prolylglycine ethyl ester.

4. The composition of claim 1 further comprising any one or more of: Panax ginseng; Ginkgo biloba; ginger root; Cordyceps militaris; Lion's Mane; L-Theanine; and Cordyceps Sinesis.

5. The composition of claim 1 wherein the racetam is aniracetam, piracetam, oxiracetam, pramiracetam, or phenylpiracetam.

6. The composition of claim 1 wherein the racetam is oxiracetam.

7. The composition of claim 2 wherein the second racetam is piracetam.

8. The composition of claim 1 further comprising psilocin or psilocybin.

9. The composition of claim 1 comprising: oxiracetam, N-phenylacetyl-L-prolylglycine ethyl ester, Panax ginseng, ginger root, Mucuna Pruriens Extract, S-adenosyl methionine, Cordyceps Militaris, Lion's Mane, L-theanine, caffeine, and Cordyceps Sinesis.

10. The composition of claim 9 comprising, as a weight percentage of active ingredients: 20-30% oxiracetam, 0.4-0.8% Noopept, 2-4% Panax ginseng, 6-8% ginger root, 10-15% Mucuna Pruriens extract, 2-5% Sam-E, 8-12% Cordyceps Militaris, 12-18% Lion's Mane, 3-5% L-theanine, 6-10% delayed release caffeine, and 8-12% Cordyceps Sinensis.

11. The composition of claim 10 comprising, as a weight percentage of active ingredients: about 25% oxiracetam, about 0.6% Noopept, about 3% Panax ginseng, about 7% ginger root, about 13% Mucuna Pruriens extract, about 3.5% Sam-E, about 10% Cordyceps Militaris, about 15% Lion's Mane, about 4% L-theanine, about 8% delayed release caffeine, and about 10% Cordyceps Sinensis.

12. The composition of claim 10 comprising, per dose: 37.5-375 mg of oxiracetam; 0.9-9 mg of N-phenylacetyl-L-prolylglycine ethyl ester; 4.3-44 mg of panax ginseng; 10.5-105 mg of ginger root; 18-190 mg of Mucuna Pruriens extract; 5-53 mg of Sam-E; 14-145 mg of Cordyceps Militaris; 22-225 mg of Lion's Mane; 6-60 mg of L-theanine; 12-120 mg of caffeine; 14-25-150 mg of Cordyceps Sinesis.

13. The composition of claim 12 comprising, per dose: 50-187.5 mg of oxiracetam; 1.2-4.5 mg of N-Phenylacetyl-L-prolylglycine ethyl ester; 5.8-22 mg of panax ginseng; 14-52.5 mg of ginger root; 25-94 mg of Mucuna Pruriens extract; 7-25 mg of Sam-E; 19-72 mg of Cordyceps Militaris; 30-112 mg of Lion's Mane; 8-30 mg of L-theanine; 16-60 mg of caffeine; and 20-72 mg of Cordyceps Sinesis.

14. The composition of claim 13 comprising, per dose: about 62.5 mg of oxiracetam; about 1.5 mg of N-phenylacetyl-L-prolylglycine ethyl ester; about 7.3 mg of panax ginseng; about 17.5 mg of ginger root; about 31.25 mg of Mucuna Pruriens extract; about 8.75 mg of Sam-E; about 23.75 mg of Cordyceps Militaris; about 37.5 mg of Lion's Mane; about 10 mg of L-theanine; about 20 mg of caffeine; and about 23.75 mg of Cordyceps Sinesis.

15. The composition of claim 1 wherein the caffeine is a delayed release caffeine.

16. The composition of claim 15 wherein the delayed release caffeine is a delayed release caffeine 60 or a delayed release caffeine 80.

17. The composition of claim 1 further comprising non-medicinally active ingredients selected from fillers, flavors, preservatives, dyes, excipients, stabilizers, sweeteners, compacting agents, solubilizing agents, anti-flocculants, and gelling agents.

18. The composition of claim 1 in the form of a gummy, a capsule, or a tablet.

19. Method of improving alertness, physical endurance, sleep patterns, memory, perceptual acuity, and/or increasing energy in an individual in need thereof, comprising administering an effective amount of a compound of claim 1 to said individual.

20. Use of a compound of claim 1 for improvement of alertness, physical endurance, sleep patterns, memory, perceptual acuity, and/or for increasing energy.
